(19) United States
(12) Patent Application Publication
(10) Pub. No.: US 2021/0275790 A1
(43) Pub. Date: Sep. 9, 2021

(54) SYSTEMS AND METHODS FOR CARBON DIOXIDE ENHANCED DELIVERY OF TOPICAL SUBSTANCES

(71) Applicant: CRYOCONCEPTS LP, Bethlehem, PA (US)

(72) Inventors: Lincoln C. YOUNG, Bethlehem, PA (US); R. Sam NIEDBALA, Bethlehem, PA (US); Philip Michael FORMICA, Bethlehem, PA (US); Amanda DEVINE, Bethlehem, PA (US)

(21) Appl. No.: 17/186,762

(22) Filed: Feb. 26, 2021

Related U.S. Application Data
(60) Provisional application No. 62/986,092, filed on Mar. 6, 2020.

(51) Int. Cl. A61M 35/00; A61B 17/32; A61H 1/00

(52) U.S. Cl. CPC A61M 35/30; A61B 17/320068; A61H 1/00; A61M 35/003; A61H 2201/0153; A61H 2201/5012; A61B 2017/00893; A61M 2202/0225; A61H 2201/0207; A61M 2205/3334; A61M 2205/3389; A61B 2017/320004; A61H 2201/5043

(57) ABSTRACT

Systems, devices, and methods for delivery of a topical substance, including medicaments and/or cosmetic formulas, with carbon dioxide to the surface of the skin. The systems regulate the pH, temperature and viscosity of the topical substance, which provides benefits to the treatment of the skin. For beauty treatments, the systems lessen wrinkles of the skin and hydrate at the same time. For medicaments with active ingredients, the systems enhance delivery and ionic state using pH adjustment. The systems include devices that contain a reservoir of carbon dioxide, which flows through a heat control path where the temperature of the carbon dioxide is adjusted to a desired setting. The temperature-controlled carbon dioxide then mixes with the topical substance in a chamber, which is pressed against or applied to the skin to achieve the benefits.

### CROSS REFERENCE TO RELATED APPLICATION

This application claims the benefit of U.S. Provisional Application No. 62/986,092 filed on Mar. 6, 2020.

### TECHNICAL FIELD

The invention relates to portable electro-mechanical devices for medical and/or cosmetic use. More specifically, the invention relates to systems and methods for use in enhancing medicament and/or cosmetic formula benefits with carbon dioxide for delivery to a user.

### BACKGROUND

Health of skin has commonly been associated with proteins related to skin structure and cell adhesion, skin surface lipids, and intercellular lipid components in the stratum corneum such as ceramides, free fatty acids, and cholesterol, and natural moisturizing factor (NMF), which is composed of amino acids and organic acids, which are critical to improving/maintaining the physiological functions of the skin. Environmental factors such as ultraviolet light, climate—especially humidity, and changes in health status and aging can cause changes in the amount and composition of the functional molecules in the skin. These changes may include inflammation, cracking, scaling, increases in lines, dryness, loss of tone, etc. Therefore, if a mechanism that regulates the structure and function of skin, and the factors that affect this mechanism can be controlled, then stability and improvement to the health of the skin may be realized.

A number of procedures exist for treatment of the skin, including for medical and cosmetic purposes. Medications or beauty formulas are topically spread on the skin, as a cream, lotion, jelly, etc., to address directly one or more conditions. These medications and/or beauty formulas may deliver hydration, luminosity, improvement to fine lines, skin tone, firmness, damage repair, etc. Additionally, naturally occurring carbon dioxide springs have been used in the past to improve and address these health conditions.

Further, topically delivered carbon dioxide acts in a different manner than in subdermal applications. For example, carbon dioxide rich water bathing has been shown to enhance collateral blood flow in animal models. In humans, published studies comparing carbon dioxide rich water to non-carbon dioxide rich water showed that the partial pressure of oxygen (i.e., oxygenation) of tissues increased about 10% with carbon dioxide enrichment and resulted in a dramatic increase in vasodilation. These experiments, whether with animal or human subjects, utilized warm water containing the carbon dioxide. The control group using non-carbon dioxide rich water at elevated temperatures (i.e., warm water) did not show the same benefits.

No previous developments have contemplated the direct combination of carbon dioxide treatments with topical substances including beauty and health treatments.

### SUMMARY

The invention includes portable systems and methods for combining topical substance treatments with carbon dioxide treatments to compound benefits of each treatment. The invention includes devices that enhance and condition (i.e., infuse, mix, etc.) using carbon dioxide from a tank reservoir with topical substances thus adjusting/controlling the temperature to a desired level. The invention provides new systems that are easy to operate and deliver the mixture to clients' skin for treatment. The invention delivers topical substances compounded with carbon dioxide to affected skin sites.

The invention provides many additional benefits not realized by existing systems. The systems and methods of the invention provide the enhanced topical substance (i.e., the carbon dioxide with the topical substance), which thermally shocks the skin, lowers the pH of the topical substance, increases the viscosity of the topical substance, provides improved topical infusion due to application of the enhanced topical substances, and provides improved topical infusion and skin treatment through physical vibration of the dispenser. Further, the devices include ergonomically designed and manufactured actuation systems, including electro-mechanical valves and dispensers.

The invention systems include a device and a container. The invention devices include a base and a hand-held dispenser. The base holds a carbon dioxide tank reservoir and valving to turn on/off the flow of gas. When turned on, the carbon dioxide flows via tubing into a hand-held dispenser. In other embodiments, placing a carbon dioxide reservoir in the hand-held dispenser may be contemplated as an all-in-one device. The dispensers and/or bases may include a thermal regulation flow path that allows heat transfer sufficient to regulate the target temperature of the carbon dioxide, and subsequently the temperature of the delivered enhanced topical substances. The device includes processors, displays and the mechanisms to dispense the topical substance for application. The mechanisms to dispense the enhanced topical substances are in the hand-held dispenser and include a container for topical substance storage and dispensing. The containers include mixing chambers for mixing prior to dispensing the enhanced topical substances. The hand-held dispensers may also be configured to spray carbon dioxide sequential to application of the topical substances to be incorporated on the treated skin.

When activated, the carbon dioxide gas flows into the hand-held dispensers. A target temperature of the gas is determined, and the carbon dioxide gas from the reservoirs are heated to the target temperature along the flow path to a mixing chamber for the gas and the topical substance. The gas then flows into the mixing chamber at the end of the flow path to be mixed with the to be dispensed topical substance. The mixing chambers are near the dispenser tip that presses and/or is applied to the skin for topical application of the enhanced topical substances. Application to the skin may include direct flow onto the skin and/or spraying onto the skin.

The invention provides simultaneous and/or sequential delivery of a topical substance with carbon dioxide to an affected area while controlling temperature and flow rate of each of the topical substance and carbon dioxide. For example, the topical substance and carbon dioxide are mixed in one embodiment. In other embodiments, the carbon dioxide may be applied before the application of the topical substance, thus the topical substance is enhanced by pre-treatment of a clients' skin. For example, with regard to the temperature, the systems are configured to determine existing temperature, including ambient environment temperatures and temperatures of the carbon dioxide in the reservoirs, and flow rates at which the carbon dioxide is currently moving or will move through its flow path. The systems may then determine a target temperature for the carbon dioxide prior to its mixture with the topical substances. Based on this target temperature, existing temperatures, and flow rate, the system may determine how to raise the temperature of the carbon dioxide from the reservoir to the target temperature, by the time the carbon dioxide is mixed with the topical substance. The systems heat the carbon dioxide along the flow paths via heat exchanger. The heat exchangers generally use the ambient heat to raise the temperature of the carbon dioxide from the reservoirs, which are generally maintained at about −79° C.—in a liquid storage form to approximately ambient temperature. The heat exchangers have a fan which passes ambient air over the flow path. The flow paths can be aluminum or another material capable of ambient heat transfer to the carbon dioxide. The fan speed is regulated to raise the temperature of the liquid carbon dioxide to the target temperature. In some embodiments, the heating may include methods of heating which will result in a-carbon dioxide temperature at the target temperature. For example, direct heating elements in a secondary chamber and/or heating along the flow path of the carbon dioxide, recovery of waste heat of the devices, other conventional heating methods, or a combination of these heating methods.

The systems of the invention include a container, which contains the topical substance. The container includes a dispensing mechanism, which cooperates with an actuating mechanism. In one embodiment, the actuating mechanism is a stepper motor that regulates the speed of the dispensing mechanism. For example, the dispensing mechanism can be a plunger which, when actuated, pushes the topical substance out of the container. In other embodiments, mechanisms, including manual mechanisms (e.g., pump, crankset, etc.), capable of controlling the flow of the topical substance out of the container, such as, hydraulic motors, etc. may be used as the actuating mechanism. The containers may be single refillable cylinder cartridges or single-use cylinder cartridges for predetermined topical substances. Further, the cartridges may include various shapes and sizes for application. In some embodiments, the cartridges dispense enough topical substance to meet the requirements of a single treatment.

The invention provides a measured delivery of the topical substance and carbon dioxide mixture to an affected area. For example, the systems can be configured to dispense the enhanced topical substances over any time duration. Time settings as low as one second to a high setting of a continuous dispensing may be used. The dispense time determines a metered volume of enhanced topical substance which is applied. Further, the volume of enhanced topical substance may also be controlled by flow rates of the topical substance and determined by dimensions of the container, e.g., length and diameter. The amount of the applied enhanced topical substance optimizes treatment to the affected areas. Clinicians and/or cosmetologists can also use an illumination source on the tip of the hand-held dispensers to illuminate the treatment areas for accurate delivery of the enhanced topical substance.

The systems of the invention include a mixing cavity for combining topical substance and carbon dioxide to create the enhanced topical substance, and a dispenser tip which applies the enhanced topical substance. In the systems, the mixing cavities and dispenser tips may be integrated into the containers to provide optimal dispenser types for different topical substances. For example, the dispenser tips may be abrasive, i.e., include exfoliants, to provide physical abrasion for cleanser topical substances, and the dispenser tip may be smooth for burn cream topical substances to prevent harm to burned skin. Further, the mixing cavities may be larger or smaller based on optimal application volumes. For example, users of steroid creams may not want to apply large volumes of the topical substance, and thus the mixing cavity may be smaller, such as between 1 and 2 ml.

The systems of the invention include hand-held dispensers for capture of containers of topical substances by connecting the containers to the dispensers. The hand-held dispensers are connected to the bases to receive the carbon dioxide from the reservoirs in the bases. The hand-held dispensers include an actuating mechanism, heat exchanger, and a display for presenting information associated with the application of the topical substances and carbon dioxide to a patient. The displays may present system status information; advertisements for other topical substances; carbon dioxide reservoir levels; topical substance information; application duration, time of use, volume, etc. In some embodiments of the invention, various portions of the container/cartridge may be incorporated into the hand-held dispenser. For example, the dispenser tips and mixing cavities may be integrated into the hand-held dispensers and topical substances may flow from the containers to the mixing cavities. In other embodiments, the containers are refillable in the hand-held dispensers to allow full integration of the containers, dispenser tips, and mixing cavities to the hand-held dispensers.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows a perspective view of an enhanced topical substance and carbon dioxide delivery system in accordance with the invention.

FIG. 1B shows a side view of the enhanced topical substance and carbon dioxide delivery system in accordance with the invention of FIG. 1A.

FIG. 1C shows a top view of the enhanced topical substance and carbon dioxide delivery system in accordance with the invention of FIG. 1A.

FIG. 1D shows a bottom view of the enhanced topical substance and carbon dioxide delivery system in accordance with the invention of FIG. 1A.

FIG. 1E shows a front view of the enhanced topical substance and carbon dioxide delivery system in accordance with the invention of FIG. 1A.

FIG. 1F shows a rear view of the enhanced topical substance and carbon dioxide delivery system in accordance with the invention of FIG. 1A.

FIG. 2A shows a side view of a container apparatus for use by the enhanced topical substance and carbon dioxide delivery system of FIG. 1A.

FIG. 2B shows a perspective view of the container apparatus for use by the enhanced topical substance and carbon dioxide delivery system of FIG. 1A.

FIG. 2C shows a perspective exploded view of the container apparatus for use by the enhanced topical substance and carbon dioxide delivery system of FIG. 1A.

FIG. 2D shows a bottom view of a container apparatus for use by the enhanced topical substance and carbon dioxide delivery system of FIG. 1A.

FIG. 2E shows a front view of a container apparatus for use by the enhanced topical substance and carbon dioxide delivery system of FIG. 1A.

FIG. 3A shows a top-side perspective view of the container apparatus describing the insertion direction into a hand-held dispenser of the enhanced topical substance and carbon dioxide delivery system of FIG. 1A.

FIG. 3B shows a top view of the container apparatus inserted into the hand-held dispenser of the enhanced topical substance and carbon dioxide delivery system of FIG. 1A.

FIGS. 4A-4D show a number of example presentations for the display on the hand-held dispensers of the enhanced topical substance and carbon dioxide delivery systems in accordance with the invention.

FIG. 5 shows an example implementation of the system electronics of the invention.

FIG. 6 illustrates the enhanced topical substance and carbon dioxide delivery system in accordance with the invention.

### DETAILED DESCRIPTION

The portable electro-mechanical devices of the invention provide applications of topical substances infused/enhanced with carbon dioxide, to affected patient skin for treatment.

### System Components

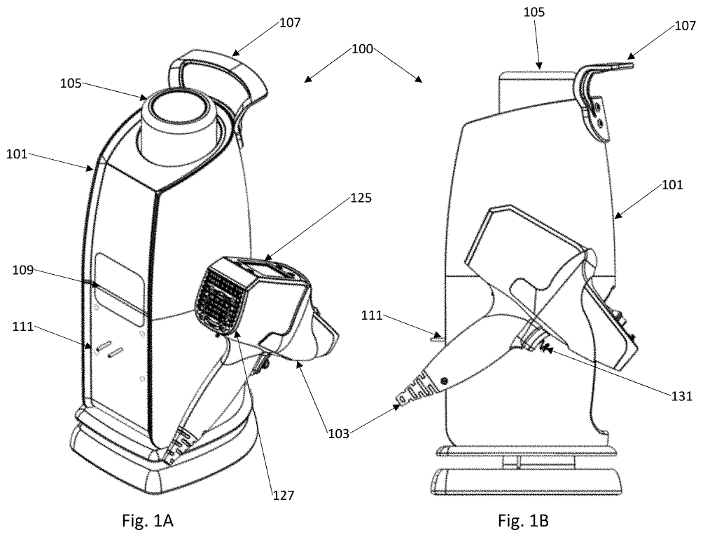

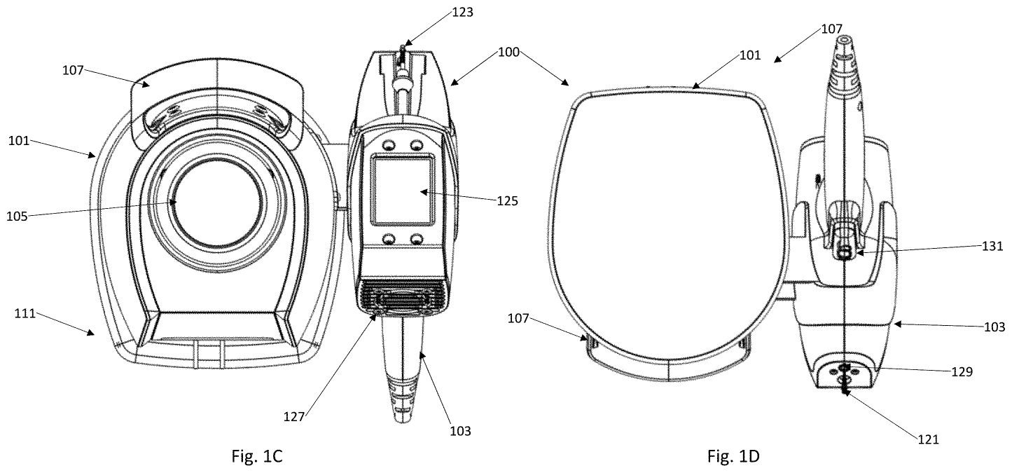

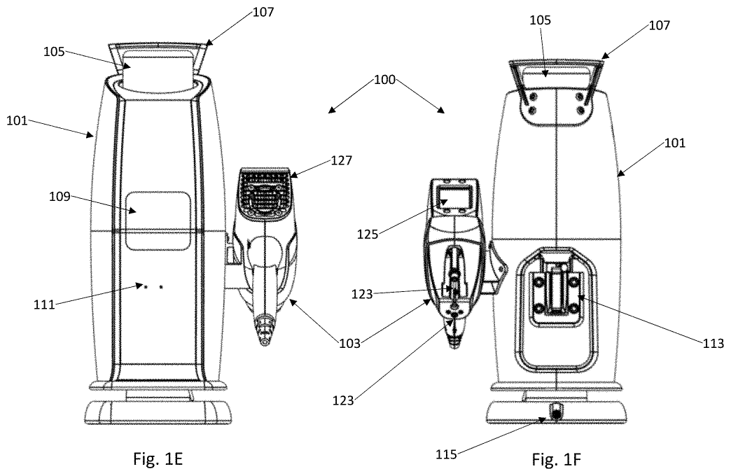

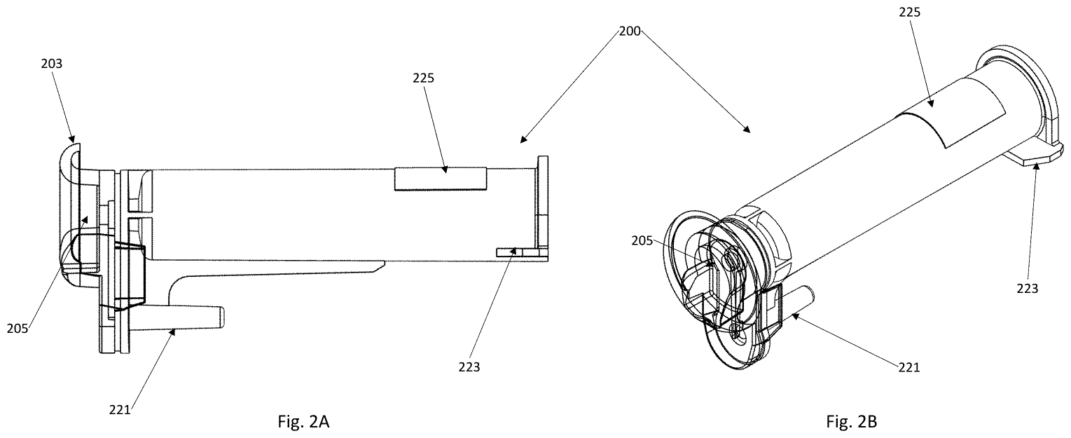

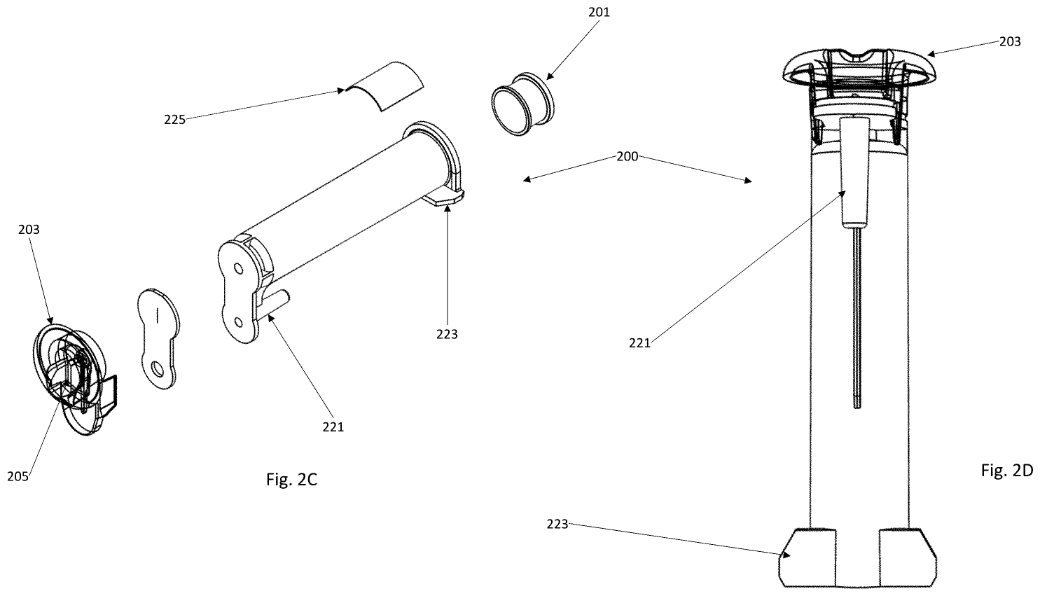

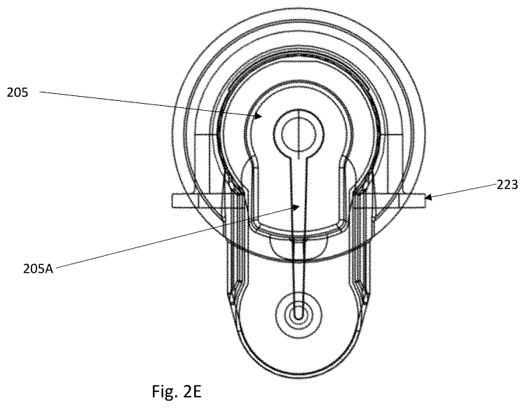

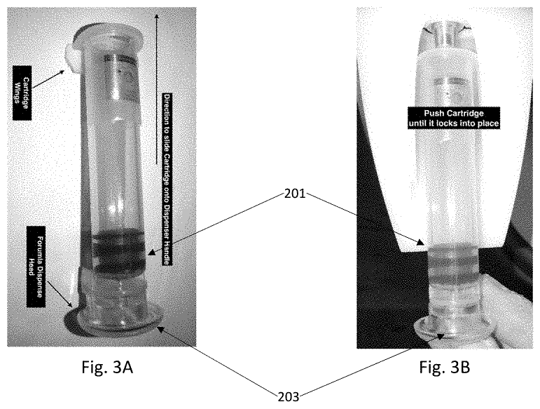

As shown in FIGS. 1A-1F, the system 100 includes a base unit 101 and a hand-held dispenser 103. FIGS. 2A-2E show a cartridge 200 configured to mate with the hand-held dispenser 103. FIGS. 3A-3B show the cartridge 200 and orientation and direction of mating with the hand-held dispenser 103. Base unit 101 houses a carbon dioxide supply in a tank reservoir 105, includes a handle 107, faceplate 109, and control panel 111 with a connection to the hand-held dispenser 103.

### System Housing and Electronics

The invention includes many features that ensure its ease of use and facilitate human factors. As shown in FIG. 1A, the control panel 111 faces front for easy user interaction. In this configuration, a clinician and/or cosmetologist can easily see all indicators and controls. The controls activate the system 100 and serve as an on/off switch.

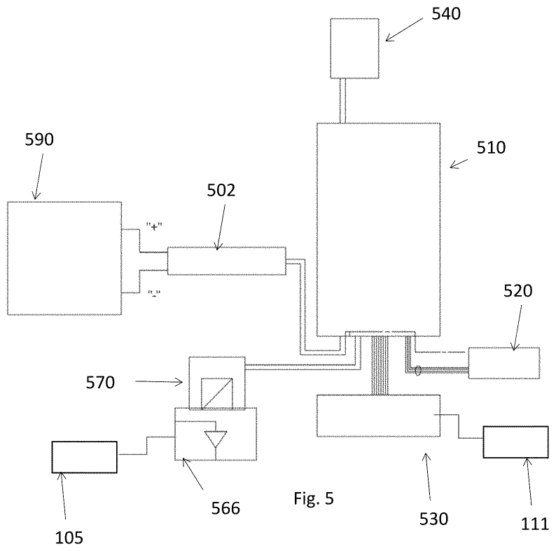

As shown in FIG. 1A, the system 100 includes a faceplate 109. The faceplate 109 may provide an indication to the clinician and/or cosmetologist of how much carbon dioxide remains in the system. As treatments are performed, the switch PCB (reference numeral 530 in FIG. 5) tracks the number of treatments and the duration of each treatment. The input PCB 510 calculates the amount of carbon dioxide used in treatments based on number and duration and subtracts it from the “full” level. The system 100 provides a visual indication of the amount of carbon dioxide remaining in the carbon dioxide tank 105 by presentation on the face plate and/or display 109, or a display of the hand-held dispenser 103.

Clinicians and/or cosmetologists can use the invention in a number of different treatment environments, including medical treatments, veterinary treatments, and aesthetic treatments. Different system configurations provide different options, depending upon the treatment environment. For example, treatment times in the medical environment differ from treatment times in the aesthetic environment. The system 100 is configured for the desired environment and topical substance.

As shown in FIG. 1F, the system 100 includes a tank lever 113. Clinicians and/or cosmetologists may insert and/or remove the carbon dioxide tank 105 by threading the 105 into the base unit 101. Once inserted, to open a sealing mechanism of the tank 105, the tank lever 113 is actuated. The lock provides an adapter connector mechanism to open flow of the carbon dioxide from the carbon dioxide tank 105 to the base 101. The handle, in a locked state, is flipped up to actuate the adapter connector mechanism, and when in an unlocked state is flipped down to release the adapter connector mechanism and re-seal the carbon dioxide tank 105. After placement of the carbon dioxide tank 105 into the base unit 101, by screwing the tank 105 into the base unit 101, access to the carbon dioxide in the tank 105 is provided by actuating the lever to a locked state.

FIG. 5 shows a diagram of an example configuration of system electronics in accordance with the invention. System electronics include an input PCB 510 connected to hand-held dispenser PCB 520. Switch PCB 530 provides connections between the input PCB 510 and user interface controls 111 shown in FIG. 1A. Input PCB 510 provides power and logic to energize solenoid coil 570 to open and close a solenoid valve and allow flow of carbon dioxide from the tank 105. System electronics are powered by power supply 590 via power connector 115 (see FIG. 1F). Power supply 590 can be a battery power supply or an AC power supply regulated to provide suitable power to input PCB 510 and to the other system electronics. A battery 540 provides power to the memory circuits when the system electronics is not fully powered.

### Hand-Held Dispenser

Clinicians and other users provide topical substances for patient treatment areas using hand-held dispenser 103 shown in FIGS. 1A-1F. The hand-held dispenser 103 includes a carbon dioxide flow connector 121, an actuating mechanism connector 123, a display 125, a heat exchanger fan 127, an illumination source 129, and an actuation button 131. The hand-held dispenser 103 includes mating portions to cartridge 200 of FIGS. 2A-2E and FIGS. 3A-3B. The mating portions include carbon dioxide flow connector 121 and actuating mechanism connector 123 to provide the cartridge with the carbon dioxide flow and a mechanism to dispense the topical substance in the cartridge 200. The actuating mechanism is integrated into the hand-held dispenser 103 to provide a mechanism to actuate a plunger of the cartridge and regulate flow of the topical substance from the containment portion of the cartridge 200. The actuating mechanism may be a stepper motor. However, other motors or other means of actuation that are capable of regulating the flow of the topical substance may be used.

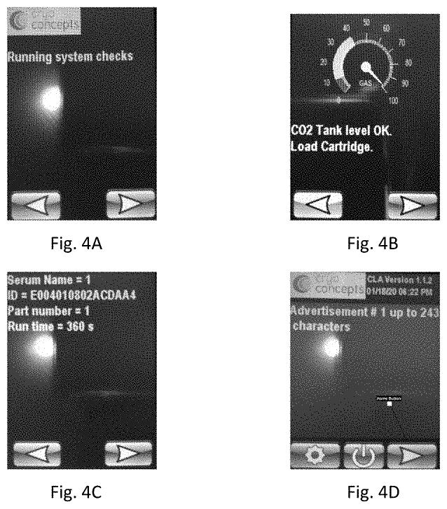

The display 125 may be used to present any information related to the application of the topical substance to a patient, or information related to the system 100 itself as described in examples shown in FIGS. 4A-4D. The heat exchanger fan 127 brings ambient air into the hand-held dispenser 103 to control the temperature of the carbon dioxide from the carbon dioxide tank 105 and provides a target temperature for application to the patient.

The hand-held dispenser 103 fits ambidextrously and comfortably into a user's hand and, because the invention includes a closed flow architecture, the wands are omni-directional, allowing clinicians to treat lesions in any direction or orientation. As shown in FIG. 1D, treatment activation button 131 is positioned on the hand-held dispenser 103 so it can be activated with a light force from either the user's index finger. As shown in FIG. 1D, the hand-held dispenser 103 includes an illumination source 129, such as an LED light for example, for extra illumination of the treatment area if the user chooses.

### Cartridge

FIGS. 2A-2E show a container/cartridge 200 configured to mate with the hand-held dispenser 103. FIGS. 3A-3B show the cartridge 200 and orientation and direction of mating with the hand-held dispenser 103. The cartridge 200, as shown in FIGS. 2A-2E includes a plunger 201, dispenser tip 203, mixing cavity 205, carbon dioxide connection channel 205A, carbon dioxide connection mate 221, wing tips 223. The plunger 201 mates with actuation mechanism connector 123 to actuate the plunger and drive the topical substance from the containment portion of the cartridge 200. The wing tips 223 aid in the orientation of the mating and a direction for to slide the cartridge 200 onto the hand-held dispenser 103. Simultaneously, the carbon dioxide connection mate 221 is mated to the flow path of the carbon dioxide through the hand-held dispenser 103 to direct the flow of the carbon dioxide into the mixing cavity 205. The connection mate 221 directs the carbon dioxide through the cartridge 200 to mixing cavity 205 via the carbon dioxide connection channel 205A. The mixing cavity 205 includes a space for the carbon dioxide as well as the topical substance to combine and simultaneously be applied to a patient. The dispenser tip 203 provides an interface to patients' skin for supporting in the treatment provided. The cartridge may be single-use or refillable and provides a way for the system 100 to be configured and capable of use with many different types of topical treatments. For example, each cartridge 200 may be filled and identified 225 with different topical substances. Each cartridge 200 may also use an identifier 225 for tracking of usage of the cartridge. The identifier 225 may include digital and/or textual identifications. Digital identifications including use of near field communications (NFC), radio frequency identification (RFID), Bluetooth, or other digital identifiers which may include tracking of other digital data. Textual identifications may include labeling such as QR codes and/or human-readable text for simple differentiation of the topical substance in the cartridge. FIGS. 3A shows the direction/orientation of the cartridge 200 as mated with the hand-held dispenser 103 and FIG. 3B shows the cartridge 200 when mated with the hand-held dispenser 103.

### System Portability

As shown in FIG. 1A, a user can carry the system 100 of the invention by the handle 107 located at the top of the base unit 101. The design and manufacture of the system 100, including the center of mass of the base unit 101, ensures that the unit is steady and hangs as expected under the handle 107 when a user lifts the unit.

### System Power Supply

An external, low voltage power supply (reference numeral 590 shown in FIG. 10) provides power to the system 100. As shown in FIGS. 1F and 5, a power connector 115 is located on the back of the system (rear of base unit 101). In one example configuration, the power supplied by the external supply also maintains an internal battery (shown as reference numeral 540 in FIG. 5) for electronics memory (not shown separately) which keeps track of carbon dioxide levels in the tank 105. The system 100 may present the user carbon dioxide tank levels as well as further instructions for use, e.g., “Load cartridge” on display 125, as shown in FIG. 4B. In one example configuration, the entire system is powered by an external battery providing additional portability and ease of use.

### System Internal Features

Internal to the base unit 101, the system 100 includes a carbon dioxide source and a flow path from the carbon dioxide source to the hand-held dispenser 103. The carbon dioxide tank 105 has been developed to be durable, convenient to use, shippable, and provides an interface that is manufactured in high volume and for proven reliability. The carbon dioxide tank 105 stores a volume of liquid carbon dioxide for shipping and storage. The carbon dioxide source tank 105 is housed inside the rear of the base unit 101 and connects to the flow path through a pin valve (not shown separately), installed in the carbon dioxide source tank 105, that seals the carbon dioxide source tank 105 during shipping and storage. An adaptor connector engages and opens the pin valve in the carbon dioxide source tank 105 when the lock handle is actuated. The adapter connector works in tandem with a burst disk (not shown separately) on the pin valve of the carbon dioxide source tank 105 that controls overpressure, providing additional safety measures. The carbon dioxide source tank 105 interfaces with a main carbon dioxide supply valve via a thread and O-ring combination (not shown separately). When base unit 101, the top of the tank 105 protrudes only minimally above the handle 107 of the base unit 101 to both mechanically protect the tank 105 and to limit the torque a user can apply when threading the tank 105 to the main carbon dioxide supply valve. An adapter connector controlled by lever 113 is part of the flow path and engages at a neck of the tank 105 to deliver the carbon dioxide to the rest of the flow path. When activated, the lever 113 opens the pin valve in the tank 105.

A user does not need any special tools to exchange carbon dioxide tank 105. The mating threads of the carbon dioxide tank 105 pin valve thread with the mating threads of the base unit 101, and the combination assembly can be quickly changed out when the carbon dioxide is depleted. A solenoid valve between the main flow path and carbon dioxide tank 105 is normally closed, which prevents the carbon dioxide from dispensing until the clinician and/or cosmetologist initiates treatment.

To open the carbon dioxide tank 105 to allow the flow of carbon dioxide through the flow path, a user engages lever 113, which in turn pushes an adaptor connector (e.g., a post) in the pin valve assembly that opens the carbon dioxide tank 105 pin valve. Once a user engages the lever 113, the carbon dioxide from the tank 105 begins to flow through the flow path to the solenoid valve. The specifically designed high pressure electro-mechanical solenoid valve provides treatment time control. The solenoid valve receives the carbon dioxide in the flow path for release when triggered.

The solenoid valve is triggered by the activator button 131 to allow carbon dioxide to flow for a predetermined time. Users can determine and predetermined times based on the clinical environment, patient needs, and topical substance.

The solenoid valve operates consistently and effectively at the pressures exerted by the carbon dioxide. The valve seal (not shown separately) is designed and manufactured to avoid swelling and failure when exposed to the liquid carbon dioxide. That is, the valve seal maintains its original geometry when exposed to the liquid carbon dioxide. While components of any valve (body, bonnet, stem, stem seals, ball, seats, etc.) will contract and expand at different rates because of different material composition or the amount of time exposed to the carbon dioxide, the solenoid valve provides a positive seal that inhibits carbon dioxide flow when the valve is closed.

Once the carbon dioxide flows through the solenoid valve, the carbon dioxide leaves the base unit 101 and moves through the flow path to the hand-held dispenser 103 of the system 100. Once the carbon dioxide travels in the hand-held dispenser 103, a heat exchanger (not shown separately) is used along the flow path to raise the temperature of the carbon dioxide to a target temperature. The system 100 may regulate the temperature by determining an ambient temperature used to raise the temperature of the carbon dioxide from around −79° C. to ambient temperature, and use the heat exchanger fan 127 to raise the carbon dioxide temperature from the reservoir tank 107 before mixing in the cartridge 200.

### Global System Management

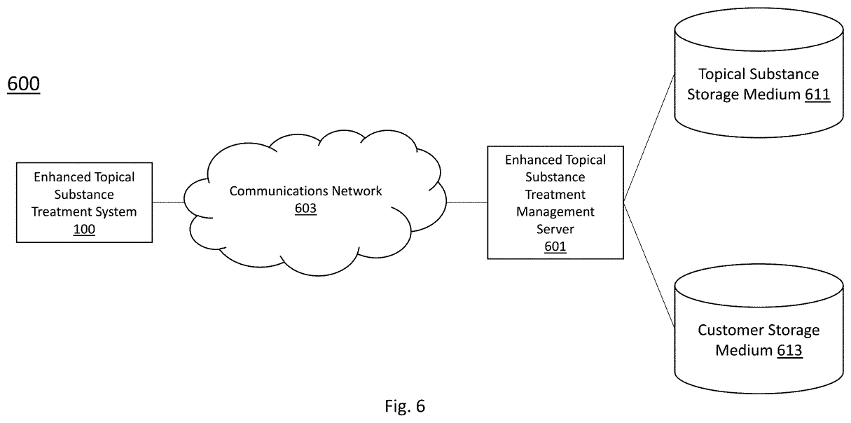

As shown in FIG. 6, the global system 600 may also provide a server 601 for management and control of a multitude of enhanced topical substance treatment systems 100. Each system 100 connects to a management server 601, via a communications network 603. The systems 100 are connected to the management server 601 for maintenance and support. The server 601 provides system 100 checks to verify proper operation, and/or to notify the management server 601 to provide maintenance and repairs to system 100, as shown in FIG. 4A. Additionally, the system 100 may provide system information such as a unit id, part numbers, and/or run time to the user as well as to the server 601, as shown in FIG. 4C. Further, some users may be provided limited features based on subscription status. For example, should a user have an expired account, based on limited uses/time or payment/billing, their user system 100 may be deactivated. Additionally, the communication connection may provide users with customized support, remote troubleshooting of equipment, firmware updates and language updates.

The management server 601 may retrieve from a topical substance storage medium 611, information associated with topical substances use and other information about the system. For example, a user may scan or provide the system with a cartridge which includes a topical substance which also has an RFID tag. The scan of the RFID tag provides all information needed by the unit for a treatment, e.g., treatment times, use of vibration, etc. This input may occur either through user input of the name, reading of a RFID as an identifier 225 incorporated with the cartridge 200, or through another scanning and/or receipt of a tagged identifier 225 on cartridge 200 (e.g., as a QR code or with an NFC tag respectively). The system 100 may then be correctly configured to apply a topical substance. Further examples may include the management system 601 tracking a tagged serial number of a topical substance cartridge 200, which shows the cartridge 200 was used a month ago.

Further, the server 601 may retrieve from a customer storage medium 613, information relating to treatments conducted. As previously stated, users may have different subscription levels which limit features of the base unit 101 and hand-held dispenser 103. Finally, the server 601 may track uses for billing purposes, and charge users per use.

Additionally, the server 601 may provide advertisements to be presented on display 125, as shown in FIG. 4D.

### System Set Up

The invention provides improvements in operation over existing topical treatment systems in both usability of the device and reliability of the system. For example, the base unit 101 and hand-held dispenser 103 ship separated from carbon dioxide tank 105. When setting up and configuring the system 100, the user (i.e., clinician and/or cosmetologist) places the carbon dioxide tank 105 in the rear of the base unit 101. The user threads the carbon dioxide tank 105 into place until its rotation stops. Once the carbon dioxide tank 105 is in place, the user raises the tank lever 113 to activate the adapter connector mechanism and enable the flow of carbon dioxide.

The lever 113 action opens the carbon dioxide tank 105 pin valve (not shown separately) and pressurizes the carbon dioxide flow path. The user then connects the system power supply (not shown separately). Once power is applied to the system, the user checks the carbon dioxide level by actuating the trigger button to determine the volume of carbon dioxide remaining in the tank 105. Further, the system 100 may include an internal memory to track the usage of the carbon dioxide and show an approximate amount of carbon dioxide remaining in the carbon dioxide tank 105. Finally, the user loads a cartridge with a topical substance for mixing with the carbon dioxide. The user slides the cartridge 200, wing tip 223 first, into contact with the mating portions of the hand-held dispenser 103 including the carbon dioxide flow connector 121 and actuation mechanism connector 123.

### Topical Substance Treatments

When the user is ready to begin a treatment, the user connects the system 100 to external power supply (not shown separately). The user activates the system by either actuating the activator button 131 or using the controls on display 125 or face plate 109. Then, the user may then actuate the activator button 131 to treat the patient as needed. However, the system 100 may also include a pre-determined treatment time based on the topical substance to prevent use of expired or other time limitations. As the activator button 131 is pressed, the carbon dioxide and topical substance from the cartridge 200 are mixed in the mixing cavity 205 of the cartridge and may simultaneously be pressed or applied to an affected area of the patient's skin.

In other embodiments, once the user presses the treatment activator button 131 on the hand-held dispenser 103, the system 100 will deliver the infused topical substance to the treatment area for a selected treatment time. For example, a user may pause the treatment to provide timed breaks between application to either let the affected skin rest or for cleansing between applications.

The invention addresses design and ease of use difficulties of many previously available topical substance application systems. The invention provides an economical and easy to use platform when performing a large number of topical treatments.

We claim:

1. A portable enhanced medicament and cosmetic formula dispenser device comprising:
the device comprises a carbon dioxide source and a temperature regulator that controls a temperature of the carbon dioxide; and
a cartridge having a topical substance including at least one of a group of the medicament and the cosmetic formula for enhancing with the carbon dioxide to create a carbon dioxide enhanced topical substance,
wherein the device applies the enhanced topical substance to a client.

2. The portable enhanced medicament and cosmetic formula dispenser device of claim 1, wherein the device further comprises:
a base unit of the device contains the carbon dioxide source; and
a hand-held dispenser of the device contains the temperature regulator.

3. The portable enhanced medicament and cosmetic formula dispenser device of claim 1, wherein the temperature regulator raises or lowers the temperature of the carbon dioxide from the carbon dioxide source to a target temperature.

4. The portable enhanced medicament and cosmetic formula dispenser device of claim 3, wherein the temperature regulator controls the temperature based on an ambient environment temperature.

5. The portable enhanced medicament and cosmetic formula dispenser device of claim 3, wherein the temperature regulator controls the temperature based on predicted waste heat from the device.

6. The portable enhanced medicament and cosmetic formula dispenser device of claim 1, wherein the enhancement is a simultaneous mixing during application on the client.

7. The portable enhanced medicament and cosmetic formula dispenser device of claim 1, wherein the enhancement is a sequential application of the carbon dioxide and the topical substance during application on the client.

8. The portable enhanced medicament and cosmetic formula dispenser device of claim 1, wherein a volume of the enhanced topical substance applied is metered based on a predetermined dispense time.

9. The portable enhanced medicament and cosmetic formula dispenser device of claim 8, wherein the volume of the enhanced topical substance delivered is further metered based on a flow rate of the topical substance.

10. The portable enhanced medicament and cosmetic formula dispenser device of claim 9, wherein the flow rate of the enhanced topical substance is based on at least one of a group of the length of the cartridge and the inside diameter of the cartridge.

11. The portable enhanced medicament and cosmetic formula dispenser device of claim 3, wherein the temperature of the carbon dioxide is metered by a flow rate of the carbon dioxide from the carbon dioxide source to the hand-held dispenser.

12. The portable enhanced medicament and cosmetic formula dispenser device of claim 3, wherein the temperature regulator is a heat exchanger using ambient temperature to raise a temperature of the carbon dioxide to the target temperature.

13. The portable enhanced medicament and cosmetic formula dispenser device of claim 1, wherein the temperature regulator heats a flow path of the carbon dioxide between the carbon dioxide source and a dispenser tip of the cartridge.

14. The portable enhanced medicament and cosmetic formula dispenser device of claim 1, wherein the cartridge further comprises:
a mixing cavity to provide a volume for the topical substance and carbon dioxide to mix.

15. The portable enhanced medicament and cosmetic formula dispenser device of claim 14, wherein the cartridge further comprises:
an actuation mechanism connector and carbon dioxide connector mating with the hand-held dispenser.

16. The portable enhanced medicament and cosmetic formula dispenser device of claim 1, wherein the device further comprises:
a vibration mechanism to abrade and/or massage the client during application.

17. A method for enhancing delivery of a topical substance including at least one of a group of a medicament and a cosmetic formula, the method comprising:
enhancing the topical substance with carbon dioxide during and prior to application to a client to create an enhanced topical substance; and
applying the enhanced topical substance to the client.

18. The method of claim 17, wherein the enhancement of the topical substance with the carbon dioxide lowers a temperature of the topical substance and increases a viscosity of the topical substance.

19. The method of claim 17, wherein the enhancement of the topical substance with the carbon dioxide lowers a pH of the topical substance.

20. The method of claim 17, wherein the enhancement of the topical substance comprises a mixing of the topical substance with the carbon dioxide.
